(19) United States
(12) Patent Application Publication
(10) Pub. No.: US 2026/0137954 A1
(43) Pub. Date: May 21, 2026

(54) OPTICAL COCHLEA IMPLANT

(71) Applicants: Georg-August-Universität Göttingen Stiftung Öffentlichen Rechts, Universitätsmedizin, Göttingen (DE); OptoGenTech GmbH, Göttingen (DE)

(72) Inventors: Tobias Moser, Göttingen (DE); Lukasz Jablonski, Göttingen (DE); Christian Goßler, Göttingen (DE)

(73) Assignees: Georg-August-Universität Göttingen Stiftung Öffentlichen Rechts, Universitätsmedizin, Göttingen (DE); OptoGenTech GmbH, Göttingen (DE)

(21) Appl. No.: 19/367,606

(22) Filed: Oct. 23, 2025

Related U.S. Application Data
(63) Continuation of application No. PCT/EP2024/060936, filed on Apr. 22, 2024.

(30) Foreign Application Priority Data
Apr. 24, 2023 (EP) 23 169 563.6

(51) Int. Cl. A61N 5/06; A61N 5/067

(52) U.S. Cl. CPC A61N 5/0622; A61N 5/0603; A61N 5/067; A61N 2005/0605; A61N 2005/0626; A61N 2005/0652

(57) ABSTRACT

An optical cochlea implant has several laser diodes which are assigned to different sound frequencies and several optical wave guides. A controller activates the laser diodes as a function of a signal describing a sound frequency spectrum in order to generate laser light and to couple the laser light into different ones of the optical wave guides which are designed such as to be suitable for guiding the laser light into different light-sensitive areas of a cochlea. The laser diodes are each activated in several separate pulselets, which individually do not result in a neural activation of the respective light-sensitive area of the cochlea. The duty cycles of the pulselets are not more than 25 %; and the pulselets in which different laser diodes are activated are interleaved in such a way that no more than a quarter of the laser diodes are activated simultaneously.

### CROSS REFERENCE TO RELATED APPLICATIONS

This application is a continuation of International Application PCT/EP2024/060936 with an international filing date of April 22, 2024 and claiming priority to European Patent Application No. EP 23 169 563.6 entitled "Verfahren für den Betrieb eines optischen Cochlea-Implantats und optisches Cochlea-Implantat zur Durchführung des Verfahrens", filed on April 24, 2023, the disclosures of which are hereby incorporated herein by reference in their entirety.

### FIELD OF THE INVENTION

The present invention relates to a method of operating an optical cochlea implant and to an optical cochlea implant suitable for carrying out the method. In particular, the invention relates to a method having the features of the preamble of claim 1 and to an optical cochlea implant having the features of the preamble of claim 9.

### BACKGROUND OF THE INVENTION

International application publication WO 2017 / 011396 A1 describes methods of frequency-modulated phase coding (FMPC) for electrical cochlear implants and cochlear implants which implement these methods. A signal describing a sound frequency spectrum is divided into several frequency bands and different bins are assigned to each frequency band. Each of the different bins is assigned to an energy level of the frequency spectrum within the respective frequency band within a period of time. For each bin, a pulse is generated in an electrode assigned to the frequency band associated with the bin if certain conditions are met with respect to the bin. Each frequency band corresponds to a channel and each frequency band is assigned to an electrode of an electrode array. The known method encodes acoustic information into electrical pulses. The stimulation can take place simultaneously at all electrodes of the electrode array, the number of independent channels being up to 22. The average pulse repetition rates in each channel are approximately 100 to 300 Hz. Volume is encoded by the number of pulses in each channel and the total number of pulses across the channels and does not require an increase in current in each channel. The low average pulse repetition rate for each channel and the fact that volume increase is not encoded by an increase in current amplitude reduce the energy consumption of the cochlear implants by a factor of approximately 5 to 10. In practice, the acoustic signal is received with a microphone. A series of filters extracts the acoustic information into the frequency bands. A short-time Fourier transform is applied to each frequency band to extract the envelope of the time signal. A pulse generator generates sequences of pulses for each frequency band with random time periods between the pulses, while the average pulse rate is varied and proportional to the amplitude of the extracted envelope.

FU-YU BEVERLY CHEN ET AL.: "Pulse-Width Modulation of Optogenetic Photo-Stimulation Intensity for Application to Full-Implantable Light Sources", IEEE Transactions on Biomedical Circuits and Systems, Volume 11, Issue 1, February 2017, Pages 28-34 disclose an optogenetic stimulation of neurons by a pulsed laser diode. The laser diode is activated in pulses comprising a variable duty cycle of, for example, 50 % or 70 % and a pulse repetition frequency of 1 kHz to 1,000 kHz. The duration of the pulsed excitation is in the range of 4 ms.

DANIEL A TAFT ET AL.: "Across-frequency delays based on the cochlear traveling wave: enhanced speech presentation for cochlear implants", IEEE Trans Biomed Eng, March 2010, 57(3):596-606 describe the stimulation of an auditory nerve with the outputs of a bank of narrow-band filters in electrical cochlear implants. To improve speech understanding, they suggest desynchronizing the frequency bands as in a normal cochlea. In a normal cochlea, the neurons closer to the base of the cochlea and corresponding to higher sound frequencies are activated later than those closer to the tip of the cochlea and corresponding to lower sound frequencies. The temporal offset of the frequency bands in relation to each other is intended to make speech easier to understand because fewer stimuli go unnoticed during the selective perception of maxima.

A method of pulsed excitation of different fluorophores of a sample in multicolor fluorescence imaging, in fluorescence cross-correlation spectroscopy (Fluorescence Cross-Correlation Spectroscopy = FCCS) and in measurements of single-pair fluorescence resonance energy transfer (spFRET) is known from BARBARA K. MÜLLER ET AL.: "Pulsed Interleaved Excitation", Biophysical Journal, Research Article, Volume 89, Issue 5, Pages 3508-3522, November 2005. Pulses from different excitation light sources emitting at different wavelengths are interleaved in such a way that the fluorescence emission generated by one pulse is completed before the next excitation pulse arrives. This means that the excitation source for each detected photon is known through time coding.

In the development of optical cochlea implants, it is found that when the same temporal excitation patterns are used as in known electrical cochlea implants, a significantly higher demand for electrical power occurs. The demand for electrical power is particularly high when the optical cochlea implant is used to neurally activate different areas of the cochlea as a function of the sound frequency spectrum of a signal to be transmitted, and thus potentially several areas of the cochlea simultaneously.

There still is a need of an optical cochlea implant in which the power consumption during simultaneous neural activation of different areas of the cochlea is limited, so that very many areas of the cochlea can be neurally activated in parallel, i.e. simultaneously, without overloading an energy source of the cochlea implant.

### SUMMARY OF THE INVENTION

The present invention relates to a method of operating an optical cochlea implant comprising a plurality of laser diodes, which are assigned to different sound frequencies and activatable to emit laser light, and a plurality of optical wave guides, which are each arranged for receiving the laser light emitted by one laser diode of the plurality of laser diodes and designed such as to be suitable for guiding the laser light into one of different light-sensitive areas of a cochlea. The method comprises receiving a signal describing a sound frequency spectrum; and activating different laser diodes of the plurality of laser diodes as a function of the signal in a plurality of separate pulselets, which individually cannot result in a neural activation of a light-sensitive area of a cochlea. The step of activating further includes setting the duty cycles of the separate pulselets to not more than 25 % and interleaving the separate pulselets, in which the different laser diodes are activated, in such a way that no more than a quarter of the different laser diodes are activated at a same time.

Further, the present invention relates to an optical cochlea implant comprising a plurality of laser diodes, which are assigned to different sound frequencies and activatable to emit laser light, a plurality of optical wave guides, which are each arranged for receiving the laser light emitted by one laser diode of the plurality of laser diodes and designed such as to be suitable for guiding the laser light into one of different light-sensitive areas of a cochlea, an energy storage, and a controller configured for receiving a signal describing a sound frequency spectrum and activating different laser diodes of the plurality of laser diodes with electrical currents from the energy storage as a function of the signal in a plurality of separate pulselets, which individually cannot result in a neural activation of a light-sensitive area of a cochlea. The controller is further configured for setting the duty cycles of the separate pulselets to not more than 25 % and interleaving the separate pulselets, in which the different laser diodes are activated, in such a way that no more than a quarter of the different laser diodes are activated at a same time.

Other features and advantages of the present invention will become apparent to one with skill in the art upon examination of the following drawings and the detailed description. It is intended that all such additional features and advantages be included herein within the scope of the present invention, as defined by the claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. In the drawings, like reference numerals designate corresponding parts throughout the several views.

FIG. 1 shows the arrangement of laser diodes, microlens arrays and optical wave guides of an optical cochlea implant according to the invention.

FIG. 2 shows the arrangement according to FIG. 1 with further components of a housing of the optical cochlea implant.

FIG. 3 shows a detail of FIG. 2, an optical window between the microlens arrays and the optical wave guides being also depicted.

FIG. 4 schematically shows a group of pulselets in which one of the laser diodes of the optical cochlea implant according to FIGS. 1 to 3 is activated.

FIG. 5 illustrates the temporal sequence of the activation of several laser diodes of the optical cochlea implant according to FIGS. 1 to 3; and

FIG. 6 schematically shows the neural activation of different light-sensitive areas of a cochlea with laser light from the laser diodes of the optical cochlea implant according to FIGS. 1 to 3.

### DETAILED DESCRIPTION

In a method of operating an optical cochlea implant according to the present disclosure, several laser diodes assigned to different sound frequencies are activated as a function of a signal describing a sound frequency spectrum in order to generate laser light and couple it into different optical wave guides. The optical wave guides are intended, i.e. suitable, for guiding the laser light to different light-sensitive areas of a cochlea. The laser diodes are each activated in several separate pulselets, which individually do not or cannot result in neural activation of the light-sensitive areas of the cochlea. The duty cycles of the pulselets are not more than 25 %, and the pulselets in which different laser diodes are activated are interleaved in such a way that no more than a quarter of the total number of laser diodes are activated simultaneously.

It is understood that the sound frequency spectrum and the signal describing the sound frequency spectrum are time-variable and their variability over time encodes a substantial part of the information that is transmitted to the cochlea using the cochlear implant. The information is transmitted by neural activation of the light-sensitive areas of the cochlea in different combinations and time sequences. The neural activation is carried out by laser light from the laser diodes, which are assigned to different sound frequencies. Each individual neural activation of one of the light-sensitive areas of the cochlea is not caused by activating the respective laser diode in a single closed pulse, so that the laser diode emits a single closed pulse of laser light. Instead, the activation of the respective laser diode for the respective neural activation is carried out by a sequence of several separate pulselets, which individually would not result in neural activation of the light-sensitive area of the cochlea, but which together cause the desired neural activation. It is generally known that neural activation of a light-sensitive area of the cochlea does not require a closed light pulse, but that a sequence of pulselets of comparable light intensity and thus reduced total light energy and electrical energy is sufficient for activating the respective laser diode.

This concept is used as a starting point and applied with particularly low duty cycles of the pulselets of no more than 25 %. In addition, the pulselets, in which various of the laser diodes are activated overlapping in time, are interleaved in such a way that the total number of simultaneously activated laser diodes and thus the total electrical power required for the activation is limited. In this way, an energy storage of the cochlea implant is not overloaded even if the signal encodes a noise with high volume in all frequency ranges, which can also be described as loud white noise. Due to the maximum duty cycle of the pulselets of no more than 25 %, the pulselets of at least four laser diodes activated with an overlap in time can be interleaved in such a way that the amplitude of the electrical power required for this is no greater than when activating a single laser diode. In relation to the total number of laser diodes, the pulselets are interleaved in such a way that no more than a quarter of the laser diodes are activated simultaneously - i.e. with simultaneous pulselets. This reduces the amplitude of the electrical power when activating the laser diodes to a quarter of the maximum possible amplitude when activating all laser diodes simultaneously.

The pulselet repetition frequency of the pulselets, in which a single laser diode is activated, is usually between 5 kHz and 200 MHz and in many cases between 10 kHz and 100 MHz. The upper limit of the pulselet repetition frequency is often 10 MHz or even just 1 MHz. However, if the appropriate laser diodes and drivers are available, the pulse repetition frequency can also be higher than 200 MHz. It is understood that the pulselet repetition frequencies of all pulselets, which are interleaved in order to activate several of the laser diodes in an overlapping manner, are the same or are integer multiples of each other.

By further reducing the maximum duty cycle of the pulselets to no more than 12.5 %, the number of laser diodes that can be activated in an overlapping manner can be increased to eight without the amplitude of the electrical power having to increase as compared to the amplitude when only a single laser diode is activated.

In the method of the present disclosure, the duty cycle of the pulselets can be used to encode the volume at the sound frequency assigned to the respective laser diode. The higher the volume and thus the amplitude of the signal at the respective sound frequency, the greater the duty cycle, which, however, must not exceed the maximum duty cycle applicable to all laser diodes.

In order to transmit the information that the sound frequency spectrum comprising a particularly high amplitude at one sound frequency, i.e. is particularly loud at this sound frequency, from a minimum amplitude of the signal at this sound frequency, in addition to the laser diode assigned to this sound frequency, laser diodes assigned to neighboring sound frequencies can also be activated. As a result, the laser light is also coupled into those of the optical wave guides that are intended to guide the laser light to neighboring light-sensitive areas of the cochlea. Up to the minimum amplitude of the signal at the respective sound frequency, the amplitude can be encoded solely by the duty cycle of the pulselets in which the associated laser diode is activated.

In the method of the present disclosure, the pulselets in which one of the laser diodes is activated can be successively interleaved with the pulselets in which various other of the laser diodes are activated. In other words, the selection of laser diodes that are activated overlapping in time can vary, even across a single neural activation of a light-sensitive area of the cochlea. The successive interleaving of the pulselets for the temporally overlapping activation of changing subsets of the laser diodes can be combined with the fact that the laser diodes are activated in a sequence of sound frequencies to which the individual laser diodes are assigned, as a function of the signal describing the sound frequency spectrum. The sound frequencies can fall in the direction of the sequence, i.e. the laser diodes that correspond to higher sound frequencies are activated first, before laser diodes that correspond to lower sound frequencies are activated. This is the order in which a normal cochlea perceives different sound frequencies sequentially, i.e. with a small time delay.

The fact that the laser diodes are activated in a sequence of increasing sound frequencies as a function of the signal reverses the sequence resulting from the spatial structure of a cochlea, with which incoming sound leads to neural activation. In a cochlea, the areas that are sensitive to sound of higher frequencies are located further out and are therefore normally neurally activated earlier.

In the method of the present disclosure, the pulselets may be grouped into separate groups of pulselets, each forming one of separate effective pulses. Each of the effective pulses can cause neural activation of one of the light-sensitive areas of the cochlea. A pulse repetition frequency of the effective pulses is in the range of 50 Hz to 1,000 Hz. A duty cycle of the effective pulses is not more than 50 % and it may be not more than 25 %. The reciprocal of the pulse repetition frequency determines the temporal resolution at which the sound frequency spectrum is encoded by the pulses.

In an embodiment, no more than four and optionally no more than two of the laser diodes are activated simultaneously in the method of the present disclosure. This reduces the amplitude of the electrical power required to activate the laser diodes to four or two times the amplitude of the electrical power required to activate a single laser diode. This limitation on the number of laser diodes activated simultaneously can be combined with a concept in which diode groups of some of the laser diodes are activated separately from each other, optionally with no more than a single one of the laser diodes of each diode group being activated simultaneously. Specifically, the laser diodes can, for example, be divided into two diode groups of equal size, with each diode group comprising, for example, 16 and optionally 32 laser diodes. In this way, laser diodes can be activated for the separate neural activation of 32 or 64 light-sensitive areas of the cochlea.

In an embodiment, the laser diodes in the pulselets are each activated with an electrical current of at least 25 mA, optionally of at least 50 mA or even of at least 75 mA. Alternatively or additionally, the laser diodes in the pulselets can each be activated with an electric current of at least 80 % of the rated current of the respective laser diode. They can also be activated with an electrical current of more than 100 % of the rated current of the respective laser diode, for example with an electrical current of up to 120 % or even up to 150 % of the rated current. The relative light output of laser diodes increases with the current with which the laser diodes are activated. This also applies to currents above the rated currents with which the laser diodes can be activated in continuous wave operation without destroying them. If a laser diode - instead of being activated with a constant current - is activated in pulses with the same average electrical power, this results in a higher light yield, i.e. a higher average light output of the laser light from the laser diode. Since laser diodes can be activated with high currents for short periods without damaging them, the electrical energy required by the cochlea implant for certain neural activations can also be reduced in this way. Thus, in the method of the present disclosure, the laser diodes may be activated with currents that are above their rated current, with which they can be activated in continuous wave operation.

According to the present disclosure, an optical cochlea implant suitable for carrying out the method of the present disclosure comprises several laser diodes, several optical wave guides, which are arranged and designed as to guide laser light from one of the laser diodes into one of different light-sensitive areas of a cochlea, an energy storage and a controller. The controller activates the laser diodes assigned to different sound frequencies with electrical currents from the energy storage in order to generate the laser light and couple it into the optical wave guides. The activation is dependent on a signal describing a sound frequency spectrum. The controller is configured such as to activate the laser diodes according to the method of the present disclosure.

The efficiency at which the laser light is coupled into the optical wave guides by the laser diodes under the boundary conditions of the implantability of the optical cochlea implant has a significant influence on the energy and power budget of an optical cochlea implant. One of these boundary conditions is that the laser diodes and the electrical drivers connected to them must not only be electrically insulated, but also hermetically shielded. The shielding to be provided for this purpose can comprise a window through which the laser light is coupled into the optical wave guides. Larger losses of laser light, which is no longer available for neural activation of the cochlea, can be avoided by arranging a micro lens array between the laser diodes and the window across which at least some of the laser diodes and entrance cross sections of the associated optical wave guides are facing each other, the micro lenses of which focus the laser light from one of the laser diodes into the entrance cross section of the associated optical wave guide. Coupling is particularly effective if optical path lengths between the exit surfaces of the laser diodes and the entrance cross sections of the associated optical wave guides are limited to no more than 1.5 mm.

Another optical cochlea implant disclosed here comprises a plurality of laser diodes, a plurality of optical wave guides arranged and designed to guide laser light from a respective one of the laser diodes into one of different light-sensitive areas of a cochlea, and an optical window across which at least some of the laser diodes and entrance cross sections of the associated optical wave guides face each other. A micro lens array is arranged between the laser diodes and the window, each of the micro lenses of which focuses the laser light from one of the laser diodes into the entrance cross section of the associated optical wave guide, and optical path lengths between the exit surfaces of the laser diodes and the entrance cross sections of the associated optical wave guides are no longer than 1.5 mm. This cochlea implant, despite a large number of laser diodes and optical wave guides for neural activation of a large number of different light-sensitive areas of a cochlea, comprises a particularly compact structure and achieves a high efficiency of the coupling of the laser light from the laser diodes into the optical wave guides and thus a limitation of the electrical power required for the activation of the laser diodes, regardless of how this electrical activation of the laser diodes takes place.

In the optical cochlea implants with the micro lens array in front of the window, the optical window may comprise a thickness of 0.2 mm to 0.6 mm, optionally of 0.3 mm to 0.4 mm and thus about 0.35 mm. Further, the window may be made of sapphire. Sapphire is biocompatible, very durable and can be easily combined with a housing of the cochlea implant made of titanium. In an embodiment, the micro lens array is made from a wafer microstructured on both sides. The wafer can be aspherically structured on at least one of its sides in the area of at least some of the micro lenses in order to achieve the desired focusing of the laser light from the laser diodes into the entrance cross sections of the optical wave guides as perfectly as possible.

The laser diodes can be arranged in front of the optical window at small lateral distances of 70 µm to 150 µm. Optionally, the lateral spacing of the laser diodes is in the range of 90 µm to 110 µm, i.e. 100 µm.

A first lens surface of the microlens array facing the respective laser diode may comprise a radius of curvature in the range from 0.060 mm to 0.075 mm or exactly 0.0687 mm. A conical constant of the respective lens surface may be in the range from -2.60 to -2.50 or may be exactly -2.547. A focal length resulting from the first lens surface can be in the range from 0.14 mm to 0.16 mm or exactly 0.150 mm. A second lens surface of the microlens array facing away from the respective laser diode may comprise a radius of curvature of 0.120 mm to 0.130 mm or exactly of 0.1264 mm, a conical constant of -0.50 to -0.40 or exactly of -0.450, and a focal length of 0.35 mm to 0.50 mm or exactly of 0.433 mm. The exact values given are those of a specific embodiment of the optical cochlea implant of the present disclosure, which has been successfully tested in practice.

A working distance of the respective laser diode to the respective micro lens can then be between 0.14 mm and 0.16 mm or exactly 0.150 mm. A lens thickness of the respective micro lens can be between 0.35 mm and 0.45 mm or exactly 0.410 mm. In an embodiment, a working distance of the respective micro lens to the optical window is not greater than 0.400 mm and optionally between 0.09 mm and 0.11 mm or exactly 0.100 mm; and a working distance of the optical window to the optical wave guides is not greater than 0.200 mm and optionally between 0.09 mm and 0.11 mm or exactly 0.100 mm. The working distance between the optical window and the optical guides can be bridged with transparent epoxy resin, in which the optical wave guides are embedded and which fixes the optical wave guides relative to the optical window. The optical path lengths between the exit surfaces of the laser diodes and the entrance cross sections of the associated optical wave guides, which result from the above exact values of the specific embodiment of the optical cochlea implant of the present disclosure, are 0.15 mm + 0.41 mm + 0.10 mm + 0.35 mm + 0.10 mm = 1.11 mm.

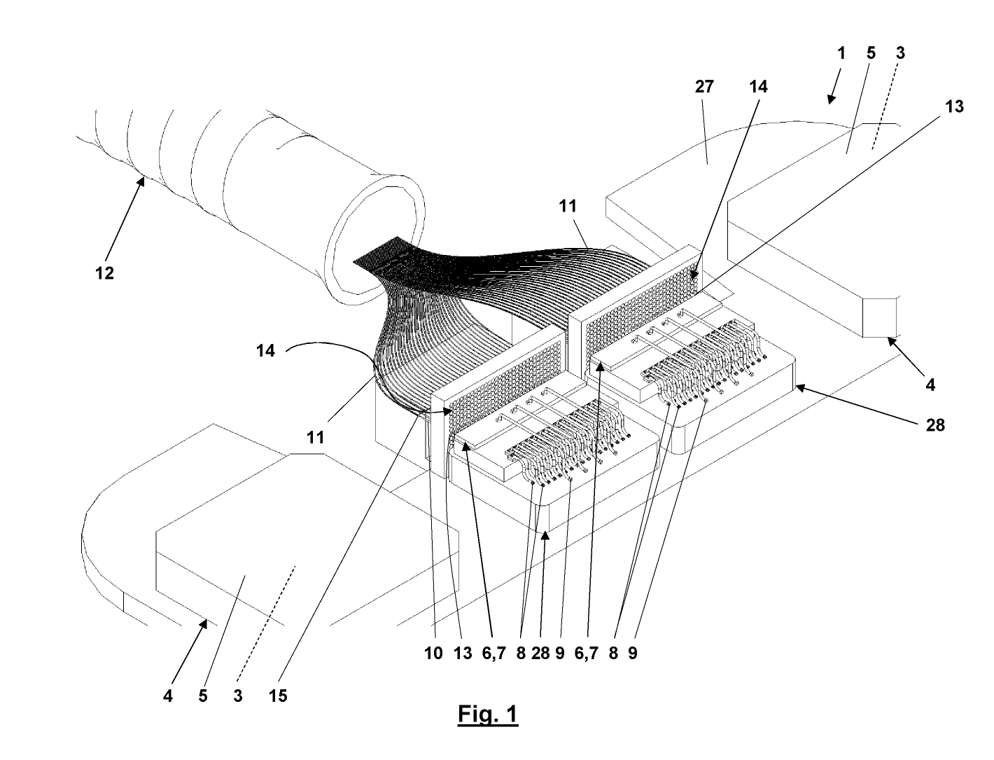

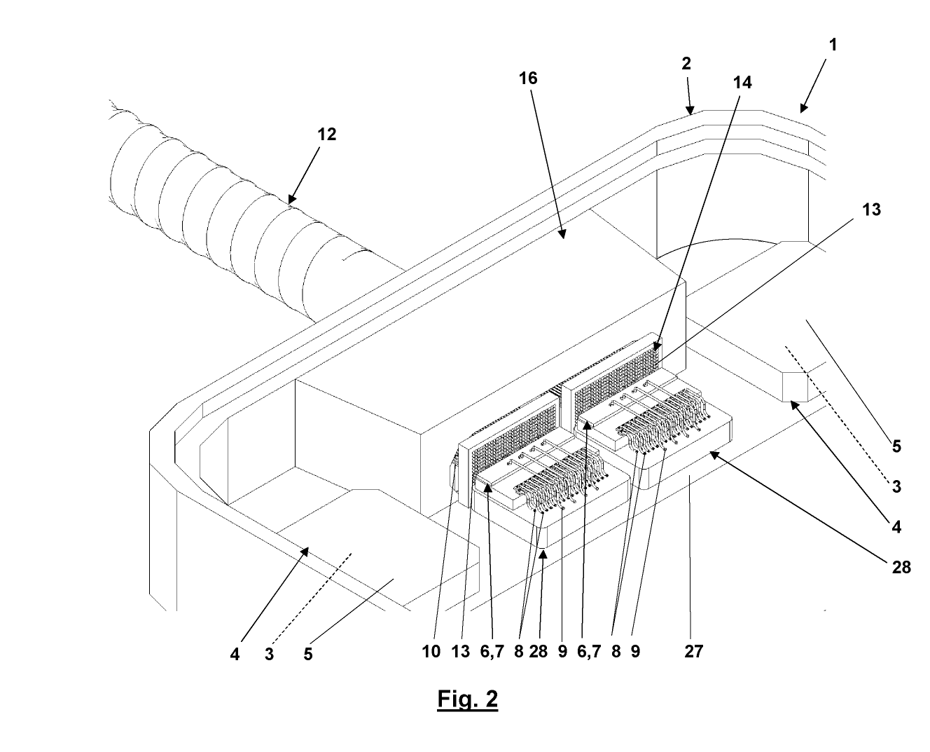

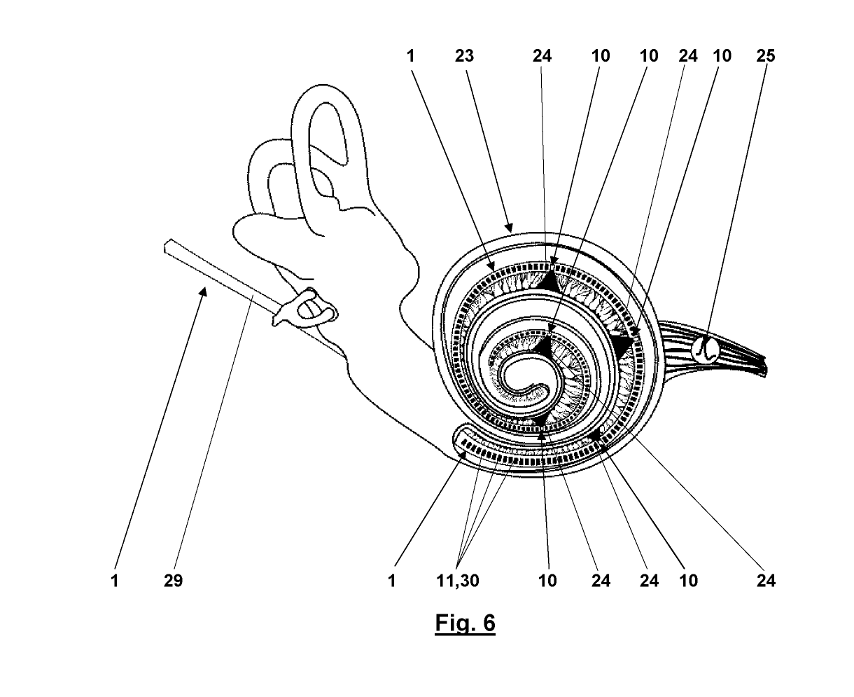

Now referring in greater detail to the drawings, the essential parts of an optical cochlea implant 1 depicted in Fig. 1 are arranged in a housing 2 made of titanium, which is only partially shown in Fig. 2. A controller 4 with integrated energy storages 3 for electrical energy is mounted on a conductor board 27 in the housing 2. The controller 4 comprises two separate drivers 5, each for a linear diode array 6 of 32 laser diodes 7. The diode arrays 6 are each mounted on the conductor board 27 via a ceramic mounting adaptor 28. The ceramic mounting adaptors 28 are important for the thermal management of the laser diodes 7. 32 control lines 8, to each of which one of the laser diodes 7 is connected, and four ground lines 9, to each of which eight of the laser diodes 7 are connected, extend from each of the drivers 5 via one of the mounting adaptors 28 to the associated diode array 6. Laser light 10 from each of the laser diodes 7 is coupled into one of 64 optical wave guides 11. The optical wave guides 11 lead into armor rods 12. The armor rods 12, which can be wound from a metal strip onto a silicon tube 29 shown in FIG. 6 and holding the optical wave guides 11, prevents the optical wave guides 11 emerging from the housing 2 from kinking. The laser light is coupled-in via a micro lens 13 of a micro lens array 14. The micro lens array 14 is a two-dimensional micro lens array. However, according to Fig. 1, only a single row of micro lenses 13 of the micro lens array 14 is used. The micro lens array is embodied as a wafer microstructured on both sides. The wafer is aspherically structured in the area of the micro lenses 13 on at least one of its sides in order to focus the laser light 10 as perfectly as possible into entrance cross sections 15 of the optical wave guides 11. An optical window arranged between the microlens arrays 14 and the entrance cross sections 15 of the optical wave guides 11 is not shown in Fig. 1. The laser diodes 7 are assigned to different sound frequencies and are activated as a function of a signal describing a sound frequency spectrum in order to couple the laser light 10 into the various optical wave guides 11. The optical wave guides 11 are intended to guide the laser light to different light-sensitive areas of a cochlea.

In addition to FIG. 1, FIG. 2 shows a connection area 16 of the housing 2, within which the optical wave guides 11 not visible here are embedded in transparent epoxy resin in order to fix them in relation to the housing 2 and thus also in relation to the optical window not shown here, so that their entrance cross sections 15 lie in the focal points of the micro lenses 13.

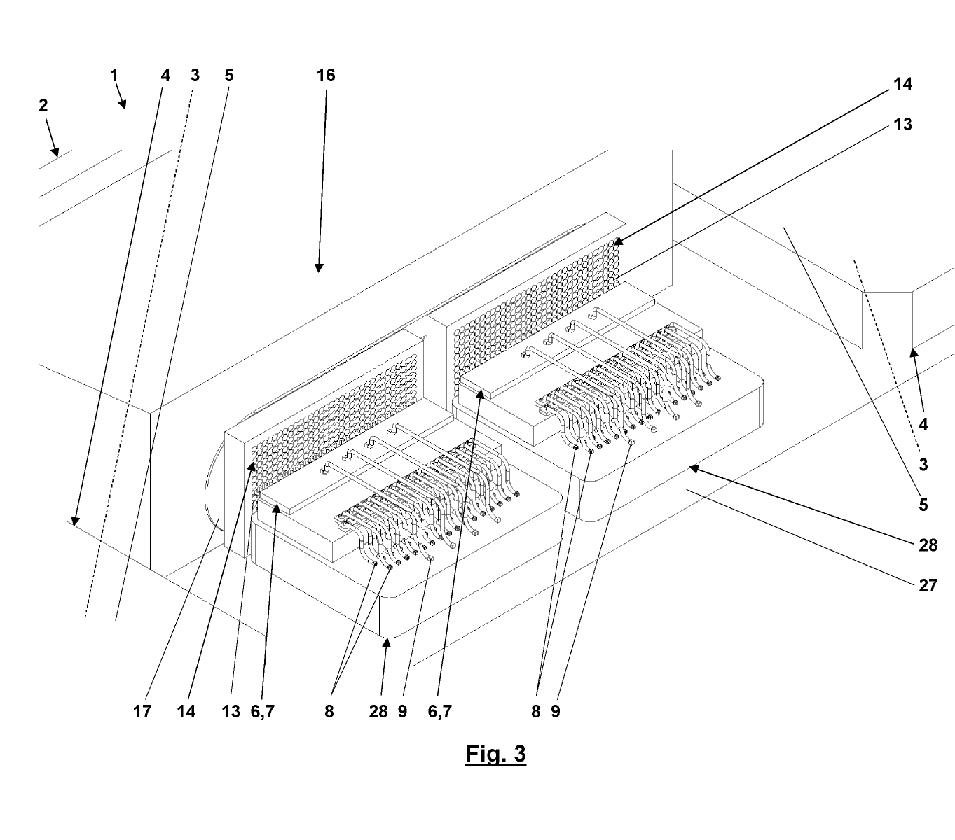

FIG. 3 shows a detail of FIG. 2, in which the optical window 17 is now shown between the entrance cross sections 15 of the optical wave guides 11 according to FIG. 1 and the microlens arrays 14.

Further details of the specific embodiment of the optical cochlea implant 1 according to FIGS. 1 to 3, which has been successfully tested in practice, are given above.

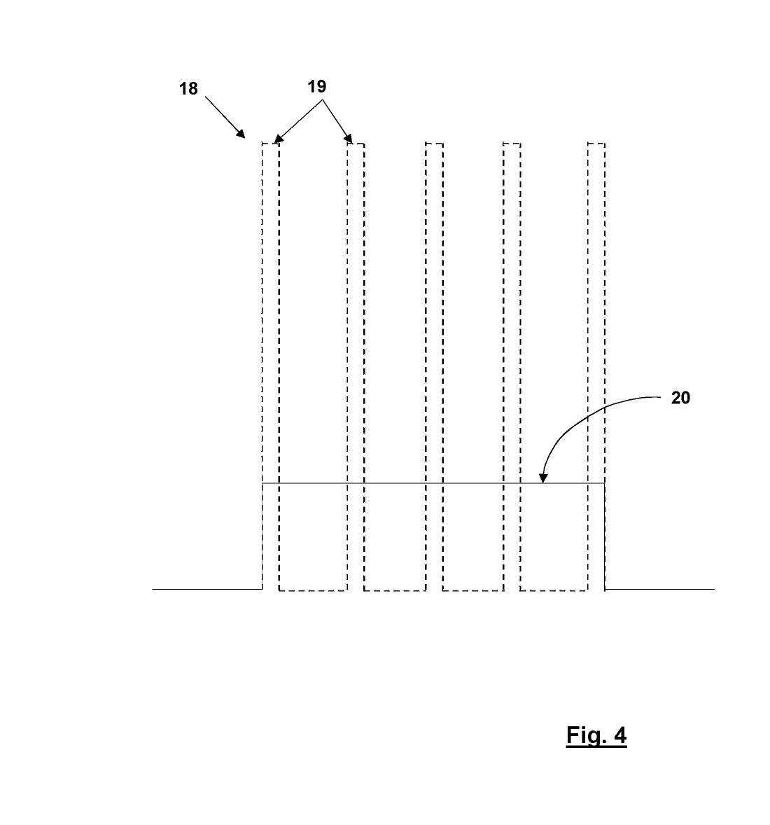

FIG. 4, with a dashed line, shows a group of pulselets 18 of individual pulselets 19 of constant amplitude and a duty cycle of about 20 %. The solid line shows a pulse 20 with a pulse energy which is exactly as large as the sum of the pulse energies of all five pulselets 19 of the group of pulselets 18. Accordingly, the constant amplitude of the pulse 20 is only one fifth as large as the amplitude of the individual pulselets 19. If the laser diodes 7 are activated by the pulselets 19 of the group of pulselets 18 instead of in a pulse 20, the ratio between the light intensities of the laser light during the pulselets 19 and during the pulse 20 is even greater, because the relative light output of laser diodes increases with the current with which the laser diodes are activated. In addition, the effect of neural activation of light-sensitive nerve cells increases with the temporal concentration of light energy on individual light pulses, even if the individual light pulses are not sufficient for neural activation, i.e. the desired neural activation is not achieved by each individual pulselet 19, but only by the entire group of pulselets 18. The activation of the laser diodes 7 of the optical cochlea implant 1 in pulselets 19 arranged in groups of pulselets 18 therefore comprises several energetic advantages.

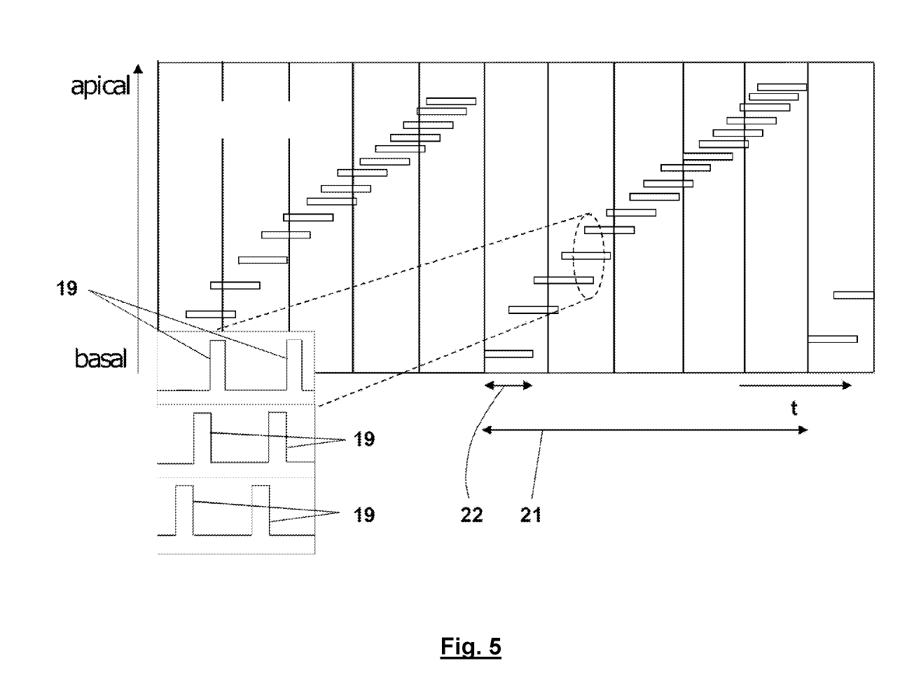

In addition, in the optical cochlea implant 1, the pulselets 19 in which various of the laser diodes 7 are activated are interleaved in time, as illustrated by way of example in FIG. 5. In FIG. 5, successive stimulation periods 21 are shown over the time t, in which different laser diodes 7 according to FIGS. 1 to 3 are each activated during a pulse duration 22 in pulselets 19 of a group of pulselets 18. The pulse durations 22 assigned to the individual laser diodes 7 are offset from one another such that the laser diodes 7 that couple their laser light 10 into the optical wave guides 11, which lead to light-sensitive areas closer to the cochlea base, i.e. basal, are activated earlier than the laser diodes 7 that couple their laser light 10 into the optical wave guides 11, which lead to light-sensitive areas of the cochlea closer to the cochlea tip, i.e. apical. Insofar as the pulse durations 22 overlap in time, the individual pulselets 19 are interleaved in such a way that two of the 16 laser diodes considered here are never activated at the same time. Pulselets 19, in which three laser diodes 7 are activated, which correspond to three adjacent light-sensitive areas of the cochlea, are shown separately. The pulselets 19 comprise a maximum duty cycle of 20 %, so that even five laser diodes 7 with interleaved pulselets 19 can be activated virtually simultaneously without two pulselets 19 overlapping in time.

FIG. 6 shows a cochlea 23 with light-sensitive areas arranged in a spiral. It is indicated that individual light-sensitive areas 24 are neurally activated by one of the laser diodes 7 by the laser light 10 emerging from a distal end of one of the optical wave guides 11 of the cochlea implant 1 in order to transmit the information corresponding to a sound frequency spectrum. The neural activations result in an electrical signal 25 that is transmitted by the auditory nerve 26. FIG. 6 shows the silicon tube 29 surrounding the optical wave guides 11 and schematically the distal ends 30 of the individual optical wave guides 11 of the cochlea implant 1.

Many variations and modifications may be made to the preferred embodiments of the invention without departing substantially from the spirit and principles of the invention. All such modifications and variations are intended to be included herein within the scope of the present invention, as defined by the following claims.

We claim:

1. A method of operating an optical cochlea implant comprising a plurality of laser diodes, which are assigned to different sound frequencies and activatable to emit laser light, and a plurality of optical wave guides, which are each arranged for receiving the laser light emitted by one laser diode of the plurality of laser diodes and designed such as to be suitable for guiding the laser light into one of different light-sensitive areas of a cochlea, the method comprising receiving a signal describing a sound frequency spectrum; and activating different laser diodes of the plurality of laser diodes as a function of the signal in a plurality of separate pulselets, which individually cannot result in a neural activation of a light-sensitive area of a cochlea; wherein the step of activating further includes setting the duty cycles of the separate pulselets to not more than 25 % and interleaving the separate pulselets, in which the different laser diodes are activated, in such a way that no more than a quarter of the different laser diodes are activated at a same time.

2. The method of claim 1, wherein the step of activating further includes providing the separate pulselets, in which the different laser diodes are activated, with a pulselet repetition frequency of 10 kHz to 10 MHz.

3. The method of claim 1, wherein the step of activating further includes at least one of setting the duty cycles of the separate pulselets as a function of an amplitude of the sound frequency spectrum at the sound frequency to which a respective laser diode of the different laser diodes is assigned, or setting the duty cycles of the separate pulselets to not more than 12.5 %.

4. The method of claim 1, wherein the step of activating further includes, from a minimum amplitude of the sound frequency spectrum at the sound frequency to which a respective laser diode of the different laser diodes is assigned, also activating laser diodes of the plurality of laser diodes which are assigned to adjacent sound frequencies.

5. The method of claim 1, wherein the step of activating further includes interleaving the separate pulselets in which one of the different laser diodes is activated successively with the separate pulselets in which varying other ones of the different laser diodes are activated.

6. The method of claim 1, wherein the step of activating further includes activating the different laser diodes in a sequence of the sound frequencies to which the individual laser diodes are assigned, the sound frequencies increasing over the sequence.

7. The method of claim 1, wherein the step of activating further includes grouping the separate pulselets in mutually separated effective pulses, each effective pulse resulting in a neural activation of one light-sensitive areas of a cochlea, the effective pulses having a pulse sequence frequency of 50 Hz to 1,000 Hz and duty cycles of not more than 50 %.

8. The method of claim 7, wherein the step of activating further includes activating, during each of the separate pulses, not more than a quarter of all laser diodes, each of the separate pulses comprising 5 to 50 pulselets per one of the different laser diodes activated.

9. The method of claim 1, wherein the step of activating further includes activating no more two of the different laser diodes simultaneously.

10. The method of claim 1, wherein the step of activating further includes activating each of the different laser diodes in the pulselets with an electric current of more than 100 % of a rated dc current of the respective laser diode.

11. An optical cochlea implant comprising a plurality of laser diodes, which are assigned to different sound frequencies and activatable to emit laser light, a plurality of optical wave guides, which are each arranged for receiving the laser light emitted by one laser diode of the plurality of laser diodes and designed such as to be suitable for guiding the laser light into one of different light-sensitive areas of a cochlea, an energy storage, and a controller configured for receiving a signal describing a sound frequency spectrum and activating different laser diodes of the plurality of laser diodes with electrical currents from the energy storage as a function of the signal in a plurality of separate pulselets, which individually cannot result in a neural activation of a light-sensitive area of a cochlea; wherein the controller is further configured for setting the duty cycles of the separate pulselets to not more than 25 % and interleaving the separate pulselets, in which the different laser diodes are activated, in such a way that no more than a quarter of the different laser diodes are activated at a same time.

12. The optical cochlea implant of claim 11, wherein the controller is further configured for providing the separate pulselets, in which the different laser diodes are activated, with a pulselet repetition frequency of 10 kHz to 10 MHz.

13. The optical cochlea implant of claim 11, wherein the controller is further configured for at least one of setting the duty cycles of the separate pulselets as a function of an amplitude of the sound frequency spectrum at the sound frequency to which a respective laser diode of the different laser diodes is assigned, or setting the duty cycles of the separate pulselets to not more than 12.5 %.

14. The optical cochlea implant of claim 11, wherein the controller is further configured for, from a minimum amplitude of the sound frequency spectrum at the sound frequency to which a respective laser diode of the different laser diodes is assigned, also activating laser diodes of the plurality of laser diodes which are assigned to adjacent sound frequencies.

15. The optical cochlea implant of claim 11, wherein the controller is further configured for interleaving the pulselets, in which it activates one of the different laser diodes, successively with the pulselets, in which it activates various other of the laser diodes.

16. The optical cochlea implant of claim 11, wherein the controller is further configured for activating the different laser diodes in a sequence of the sound frequencies to which the individual laser diodes are assigned, the sound frequencies increasing over the sequence.

17. The optical cochlea implant of claim 11, wherein the controller is further configured for grouping the separate pulselets in mutually separated effective pulses, each effective pulse resulting in a neural activation of one light-sensitive areas of a cochlea, the effective pulses having a pulse sequence frequency of 50 Hz to 1,000 Hz and duty cycles of not more than 50 %.

18. The optical cochlea implant of claim 11, wherein the controller is further configured for activating, during each of the separate pulses, not more than a quarter of all laser diodes, each of the separate pulses comprising 5 to 50 pulselets per one of the different laser diodes activated.

19. The optical cochlea implant of claim 11, wherein all the laser diodes of the plurality of laser diodes are divided up into two diode groups with separate drivers, wherein the controller is further configured for activating only one laser diode of each of the diode groups at one time.

20. The optical cochlea implant of claim 11, wherein the controller is further configured for activating each of the different laser diodes in the pulselets with an electric current of more than 100 % of a rated dc current of the respective laser diode.
